(19) United States
(12) Patent Application Publication
(10) Pub. No.: US 2022/0168469 A1
(43) Pub. Date: Jun. 2, 2022

(54) OBTURATOR, METHODS OF FORMING A PREFABRICATED, INNERVATED, PRE-VASCULARIZED, PRELAMINATED (PIPP) FLAP USING AN OBTURATOR TO MAINTAIN A STOMA OR LUMEN, AND METHODS OF RESTORING DAMAGED OR SURGICALLY-REMOVED SOFT TISSUE WITH A PIPP FREE OR ROTATIONAL FLAP

(71) Applicant: The Regents of The University of Michigan, Ann Arbor, MI (US)

(72) Inventors: Stephen E. FEINBERG, Ann Arbor, MI (US); Shiuhyang KUO, Dexter, MI (US); James Brian WASHINGTON, Saline, MI (US)

(21) Appl. No.: 17/540,723

(22) Filed: Dec. 2, 2021

Related U.S. Application Data
(60) Provisional application No. 63/120,568, filed on Dec. 2, 2020.

(51) Int. Cl. A61L 27/36; C12N 5/071

(52) U.S. Cl. CPC A61L 27/3695; A61L 27/362; C12N 2502/094; C12N 5/0629; C12N 2533/92; A61L 27/3633

(57) ABSTRACT

The present disclosure provides, in various aspects, a method of forming a prefabricated innervated pre-vascularized pre-laminated (PIPP) flap having a stoma or lumen. The method includes providing a cell construct including skin cells and/or mucosa cells. The method further includes forming an integrated in vivo composite at a donor site by grafting the cell construct onto a muscle. The method further includes stabilizing the composite on an obturator component. The method further includes developing a microvascular system in the composite by retaining it in vivo at the donor site for a predetermined period of time. The method further includes removing the obturator component from the stoma or lumen. In certain aspects, the present disclosure also provides a method of restoring a defect including damaged or surgically removed soft tissue using a PIPP flap. In certain aspect, the present disclosure also provides an obturator component for maintaining the stoma or lumen.

### CROSS-REFERENCE TO RELATED APPLICATIONS

This application claims the benefit of U.S. Provisional Application No. 63/120,568, filed on Dec. 2, 2021. The entire disclosure of the above application is incorporated herein by reference in its entirety.

### GOVERNMENT SUPPORT

This invention was made with government support under grant no. F056925, award no. AWD004246 awarded by the National Institutes of Health (NIH). The government has certain rights in the invention.

### FIELD

The present disclosure relates to methods of forming a prefabricated, innervated, pre-vascularized, pre-laminated (PIPP) flap using an obturator component to maintain a stoma or lumen, methods of restoring damaged or surgically-removed soft tissue with a PIPP free or rotational flap, and an obturator component for maintaining a stoma or lumen.

### BACKGROUND

This section provides background information related to the present disclosure which is not necessarily prior art.

Soft tissue defects may occur as a result of trauma, tumor ablation, or congenital deformities. In these situations, there may be a lack of healthy soft tissue available to be used to reconstruct the soft tissue defects. This is even more complex for defects in regions where multiple types of tissue compose the structure to be repaired, such as is seen with the lip, that is, epithelium from both oral mucosa and skin, dermis, and muscle. It is even more difficult not only to establish the area anatomically but also to be able to restore both function and esthetics.

### SUMMARY

This section provides a general summary of the disclosure, and is not a comprehensive disclosure of its full scope or all of its features.

In various aspects, the present disclosure provides a method of forming a prefabricated innervated pre-vascularized pre-laminated (PIPP) flap having a stoma or lumen. The method includes providing a cell construct including skin cells, mucosa cells, or both skin cells and mucosa cells. The method further includes forming an integrated in vivo composite at a donor site by grafting the cell construct onto a muscle. The method further includes stabilizing the composite on an obturator component. The obturator component is configured to maintain a stoma or lumen in the composite or formed by the composite. The method further includes developing a microvascular system in the composite by retaining the composite in vivo at the donor site for a first predetermined period of time. The method further includes removing the obturator component from the stoma or lumen.

In one aspect, the stabilizing is performed at the donor site. The developing includes retaining the composite and the obturator component in vivo at the donor site for the first predetermined period of time. The method further includes harvesting the PIPP flap by surgically removing the composite from the donor site. The PIPP flap is a free flap.

In one aspect, the stoma or lumen is the stoma. The stabilizing includes creating the stoma in the muscle. The stoma is substantially parallel to strata of fibers of the muscle. The stabilizing further includes inserting at least a portion of the obturator component into the stoma to maintain the stoma.

In one aspect, the obturator component includes a planar base portion, an obturator portion, and an elongated anchor portion. The planar base portion defines a longitudinal axis. The longitudinal axis is substantially perpendicular to a plane of the planar base portion. The obturator portion extends from the planar base portion. The obturator portion defines a first axis perpendicular to the longitudinal axis. The elongated anchor portion extends from the obturator portion. The elongated anchor portion defines a second axis perpendicular to the longitudinal axis and nonparallel to the first axis. The obturator portion is between the planar base portion and the elongated anchor portion along the longitudinal axis. The obturator portion is configured to maintain the stoma.

In one aspect, the method further includes surgically creating a PIPP rotational flap including the composite such that a neurovascular pedicle of the muscle of the composite remains intact. The stoma or lumen is the lumen. The stabilizing is performed at a recipient site of a defect and includes rolling the rotational flap around at least a portion of a length of the obturator component to define at least a portion of the lumen.

In one aspect, the obturator component includes an elongated body defining the length.

In one aspect, the obturator component further includes a flange.

In one aspect, the cell construct is a mucocutaneous construct (MCC) including a first region including skin cells and a second region including mucosa cells.

In one aspect, the providing includes creating the MCC in vitro.

In one aspect, creating the MCC includes providing a first population of skin keratinocytes and a second population of mucosa keratinocytes. Creating the MCC further includes creating a coculture by seeding the first population and the second population on a respective first portion and second portion of a non-immunogenic acellular dermal matrix in a first liquid phase. The first portion and the second portion are separated by a mechanical barrier. Creating the MCC further includes retaining the coculture with the mechanical barrier in the first liquid phase for a second predetermined period of time. Creating the MCC further includes removing the mechanical barrier and retaining the coculture in a second liquid phase for a third predetermined period of time. Creating the MCC further includes maturing and stratifying the coculture by retaining the coculture for a fourth predetermined period of time, thereby forming the MCC.

In one aspect, the providing the first population includes collecting a skin sample via punch biopsy or surgical incision. Providing the first population further includes extracting skin keratinocytes from the skin sample. Providing the first population further includes creating primary skin keratinocyte cultures from the skin keratinocytes. Providing the first population further includes amplifying a population of the primary skin keratinocyte cultures.

In one aspect, the providing the second population includes collecting a mucosa sample via punch biopsy or surgical incision. Providing the second population further includes extracting mucosa keratinocytes from the mucosa sample. Providing the second population further includes creating primary mucosa keratinocyte cultures from the mucosa keratinocytes. Providing the second population further includes amplifying a population of the primary mucosa keratinocyte cultures.

In one aspect, the mucosa keratinocytes are keratinized or non-keratinized oral mucosa keratinocytes.

In one aspect, the muscle is a latissimus dorsi muscle (LDM), a platysma muscle, or a gracilis muscle.

In one aspect, the first predetermined period of time is in a range of 10 days to 20 days.

In various aspects, the present disclosure provides a method of restoring a defect including damaged soft tissue. The method includes providing a cell construct including skin cells, mucosa cells, or both skin cells and mucosa cells. The method further includes forming an integrated in vivo composite at a donor site by grafting the cell construct onto a muscle. The method further includes stabilizing the composite on an obturator component. The obturator component is configured to maintain a stoma or lumen in the composite or formed by the composite. The method further includes developing a microvascular system in the composite by retaining the composite in vivo at the donor site for a predetermined period of time. The method further includes removing the obturator component from the stoma or lumen. The method further includes transferring the composite to a recipient site of the defect.

In one aspect, the recipient site and the donor site are on the same human or animal.

In one aspect, the skin cells, the mucosa cells, or both the skin cells and the mucosa cells are grown from the same human or animal.

In one aspect, the method further includes harvesting a PIPP free flap by surgically removing the composite from the donor site. The stabilizing is performed at the donor site prior to the developing. The developing includes retaining the composite and the obturator component in vivo at the donor site for the predetermined period of time. The transferring includes micro-anastomosing neurovascular pedicles in the PIPP free flap to respective vascular pedicles and a motor nerve at the recipient site.

In one aspect, the method further includes, after the developing, surgically creating a PIPP rotational flap including the composite such that a neurovascular pedicle of the muscle of the composite remains intact. The stoma or lumen is the lumen. The transferring includes rotation of the PIPP rotational flap around the neurovascular pedicle from the donor site to the recipient site. The stabilizing is performed at the recipient site. The stabilizing includes rolling the PIPP rotational flap around at least a portion of a length of the obturator component to form at least a portion of the lumen.

In one aspect, the transferring includes modifying a shape, a size, or both a shape and size of the composite based on the recipient site.

In one aspect, the cell construct is a mucocutaneous construct (MCC) including a first region including skin cells and a second region including mucosa cells.

In one aspect, the damaged soft tissue includes at least a portion of lips, an eyelid, an ear, a nose, a vagina, or an anal sphincter.

In one aspect, the providing includes creating the MCC in vitro.

In various aspects, the present disclosure provides an obturator component for forming a prefabricated innervated pre-vascularized pre-laminated (PIPP) free flap having a stoma. The obturator component includes a planar base portion, an obturator portion, and an elongated anchor portion. The planar base portion defines a longitudinal axis. The longitudinal axis is substantially perpendicular to a plane of the planar base portion. The obturator portion extends from the planar base portion. The obturator portion defines a first axis perpendicular to the longitudinal axis. The elongated anchor portion extends from the obturator portion. The elongated anchor portion defines a second axis perpendicular to the longitudinal axis and nonparallel to the first axis. The obturator portion is between the planar base portion and the elongated anchor portion along the longitudinal axis. The obturator portion is configured to maintain the stoma.

In one aspect, the first axis is substantially perpendicular to the second axis.

In one aspect, the planar base portion is substantially cylindrical.

In one aspect, the obturator portion is substantially elliptical cylindrical and the first axis is a major axis of the ellipse.

In one aspect, the elongated anchor portion is substantially a rectangular prism.

In one aspect, the obturator component includes a unitary structure including biocompatible silicone.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flowchart depicting a method of restoring a defect including damaged soft tissue using a prefabricated innervated pre-vascularized pre-laminated (PIPP) free flap according to various aspects of the present disclosure.

FIG. 2 is a flowchart depicting a method of manufacturing a mucocutaneous construct (MCC) in vitro according to various aspects of the present disclosure.

FIGS. 3-5B are depict steps of the method of FIG. 2; FIG. 3 is a top view of a coculture of skin and mucosa keratinocytes separated by a mechanical barrier in a first liquid phase, the coculture including two cell-free zones; FIG. 4 is a top view of the coculture without the mechanical barrier in a second liquid phase; FIG. 5A is a top view the coculture on a riser in an air-liquid phase; and FIG. 5B is a perspective view of the riser of FIG. 5A.

FIG. 6 is a schematic view of a template for lips including an MCC.

FIGS. 7-8 depict an obturator component according to various aspects of the present disclosure; FIG. 7 is a perspective view and FIG. 8 is a top view.

FIG. 9 is a sectional view of a composite stabilized on the obturator component of FIGS. 7-8 according to various aspects of the present disclosure.

FIG. 10 is a perspective view of another obturator component according to various aspects of the present disclosure.

FIGS. 11-13 are schematic views relating to transfer of a PIPP free flap from a donor site to a recipient site of a defect; FIG. 11 depicts the PIPP flap intact at the donor site; FIG. 12 depicts the PIPP free flap removed from the donor site; and FIG. 13 depicts the PIPP free flap implanted at the recipient site.

FIG. 14 is a flowchart depicting a method of restoring a defect including damaged soft tissue using a PIPP rotational flap according to various aspects of the present disclosure.

FIG. 15 is a perspective view of yet another obturator component according to various aspects of the present disclosure.

FIGS. 16-17 depict a composite stabilized on an obturator component of FIG. 15 according to various aspects of the present disclosure; FIG. 16 depicts the composite in partial perspective cutaway; and FIG. 17 is a schematic view depicting the composite at a recipient site of a defect.

FIGS. 18A-18C are photographs depicting steps of a method of manufacturing an MCC according to various aspects of the present disclosure; FIG. 18A depicts a mechanical barrier producing one cell-free zone on a scaffold; FIG. 18B depicts a corral on the scaffold without the mechanical barrier; and FIG. 18C depicts the scaffold on an insert, which is on a riser.

FIGS. 19A-19F are photographs depicting portions of a method of forming a composite, stabilizing the composite on an obturator, and removing the obturator according to various aspects of the present disclosure.

FIG. 20 depicts an immunohistochemistry of a 4-week post-implanted ex vivo-produced oral mucosa equivalent (EVPOME).

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

Certain areas of the body are difficult to reconstruct after traumatic avulsion injury or surgical resection secondary to loss of volumetric muscle mass because they represent a dynamic composite tissue of sensate mucosa, skin, and motor innervation of skeletal muscles. Tissue engineering and regenerative medicine (TE/RM) is a potential approach to repair these defects with autogenous tissue. However, TE/RM faces several barriers that prevent translation of in vitro technology to the clinical arena, such as the inability to create complex composite soft tissue structures that contain striated muscle, skin, and mucosa with a mucocutaneous junction and difficulty in developing an in vivo perfusion system (blood vessels) to supply nutrition for large segments of tissue created in vitro. Lack of tissue perfusion significantly limits survival of in vitro produced complex composite soft tissue implants.

To overcome the TE/RM barriers for functional reconstruction of the lips (composite soft tissue fabrication and vascularity/perfusion), the present disclosure provides, in various aspects, a surgical technique of pre-lamination to create a designer prefabricated innervated pre-vascularized pre-laminated (PIPP) composite soft tissue microvascular-free flap based on an undamaged muscle at a donor site (e.g., the latissimus dorsi muscle (LDM)). Lamination refers to a process of bonding of layers. Pre-lamination designates a reconstructive process whereby a three-dimensional (3D) structure is built at a remote donor site by laminating different layers of components as composite grafts into a reliable existing axial vascular bed, allowing the structure about two weeks to mature before transferring the unit en bloc to the defect based on its native axial blood supply. The technique of pre-lamination allows reconstruction to begin at a remote site in the same individual. This is advantageous because the recipient site being reconstructed may lack the blood supply or healthy tissue necessary to support construction of a sophisticated 3D construct at the defect site. Remote reconstruction in an unscarred vascular bed offers the best chance for the composite grafts to mature.

In various aspects, the present disclosure provides an obturator component for maintaining an opening in internal tissues for an extended period of time, such as while an in vivo composite develops a vascular system and a motor nerve system. The obturator may also be used to assist in stabilization of the tissues or tissue layers, in the case of pre-laminated tissue layers. Obturators may be made in custom sizes, and/or geometric shapes to accommodate tissues for a particular defect or in bulk for common procedures.

In various aspects, the present disclosure provides a method of creating functional soft tissue structures (e.g., including striated muscle, skin, and mucosa) having an intact in vivo perfusion system to supply nutrition and motor innervation to restore functionality to large segments of tissue created in vitro and implanted in situ in a human or animal body. The method includes in vitro manufacturing of a cell construct, such as a mucocutaneous construct (MCC); in vivo development of PIPP flap, such as a free flap or a rotational pedicled flap, having a stoma or lumen, respectively, from the cell construct at a donor or recipient site, respectively; and transferring of the flap to a recipient site of the defect. In certain aspects, the donor site and the recipient site are on the same individual and the cell construct is prepared from cells of the same individual. The method may be used to repair defects in human or animal lips, eyelids, ears, nose, vagina, penis, or anal sphincter, for example.

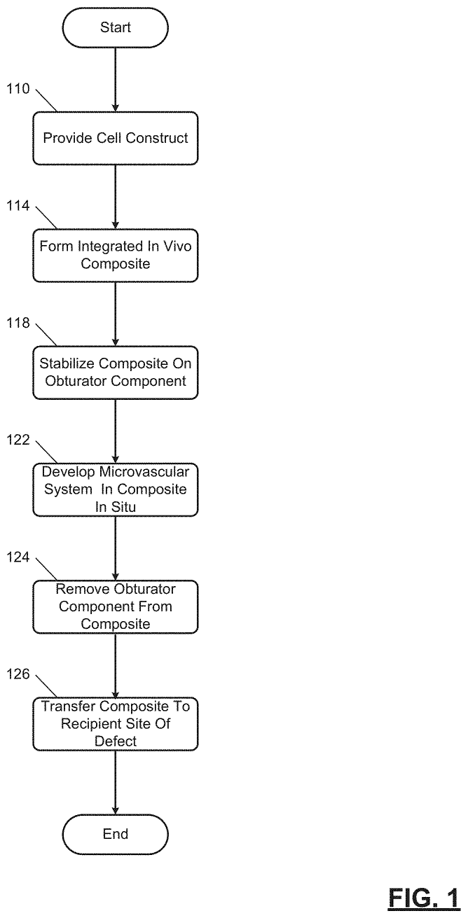

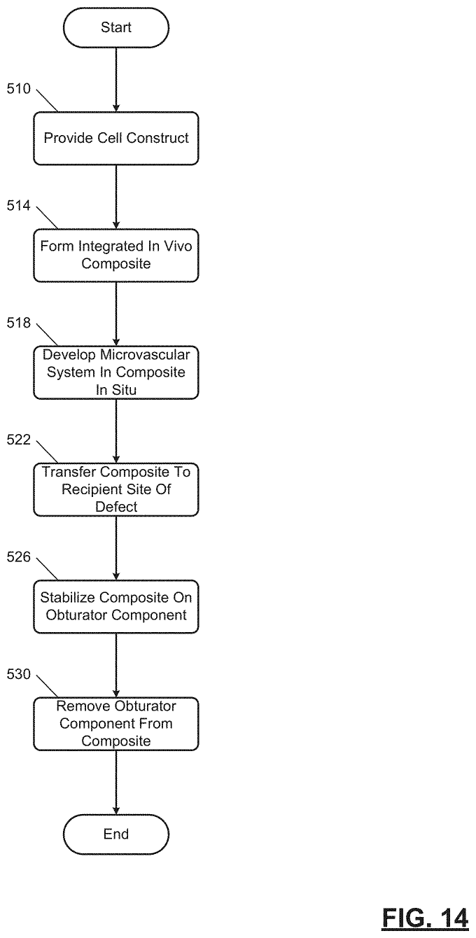

Depending on the location and type of defect, the PIPP flap may be a PIPP free flap that is completely surgically removed from the donor site and micro-anastomosed at a recipient site of the defect or a PIPP rotational flap that is rotated to the recipient site while remaining attached to a neurovascular pedicle of a muscle at the donor site. For both the PIPP free flap and the PIPP rotational flap, the method generally includes providing a cell construct (e.g., a MCC); forming an integrated in vivo composite at a donor site by grafting the cell construct onto an appropriate muscle; stabilizing the composite onto an obturator component to maintain a stoma or lumen, either at the donor or the recipient site, respectively; developing a microvascular system in the composite by retaining the composite in vivo for a predetermined period of time, and transferring the composite to a recipient site of the defect based on a native axial blood supply for the free flap and at the recipient site for the rotational flap. Example methods of restoring damaged soft tissue using a PIPP free flap and a PIPP rotational flap are described in greater detail below and depicted in FIGS. 1 and 14, respectively.

Example methods according to various aspects of the present disclosure are described in Atsuko Miyazawa, Shiuhyang Kuo, James Washington, Stephen E. Feinberg, Tissue Eng'g of Composite Soft Tissue Grafts for Craniomaxillofacial Reconstruction, in TISSUE ENG'G IN ORAL AND MAXILLOFACIAL SURGERY 71-83 (Riitta Seppanen-Kaijansinkko Ed., 2019), which is attached to this application by appendix and which is incorporated by reference in its entirety.

### Repairing a Soft Tissue Defect with a PIPP Free Flap

With reference to FIG. 1, a method of repairing a soft tissue defect with a PIPP free flap according to various aspects of the present disclosure is provided. The method begins at 110 by providing a cell construct. At 114, the method includes forming an integrated in vivo composite at a donor site. At 118, the method further includes stabilizing the composite on an obturator component. At 122, the method further includes developing a microvascular system in the composite in situ. At 124, the method further includes removing the obturator component from the composite. At 126, the method further includes transferring the composite to a recipient site of the defect. The method ends. Each of these steps is described in greater detail below.

### Providing a Cell Construct

At 110, the method includes providing a cell construct. The cell construct includes skin cells, mucosa cells, or both skin cells and mucosa cells. The skin cells and/or mucosa cells may be provided on an acellular dermal matrix (scaffold). In certain aspects, the skin cells are skin keratinocytes and the mucosa cells are mucosa keratinocytes. In certain aspects, the cell construct is a mucocutaneous construct (MCC) including a first region including skin cells and a second region including mucosa cells.

In certain aspects, providing the cell construct includes creating the cell construct in vitro. Example protocols for manufacturing an in vitro tissue-engineered mucocutaneous junction (MJ) constructs are described in Peramo A, Marcelo C L, Feinberg S E, Tissue Eng′g of Lips and Muco-cutaneous Junctions: In Vitro Dev. of Tissue Engineered Constructs of Oral Mucosa and Skin for Lip Reconstruction, TISSUE ENG PART C METHODS, 18(4):273-82 (2012); and Kuo S, Kim H M, Wang Z, Bingham E L, Miyazawa A, Marcelo C L, Feinberg S E, Comparison of Two Decellularized Dermal Equivalents, J. TISSUE ENG'G REGEN. MED., 12(4):983-90 (2018), incorporated herein by reference in their entireties.

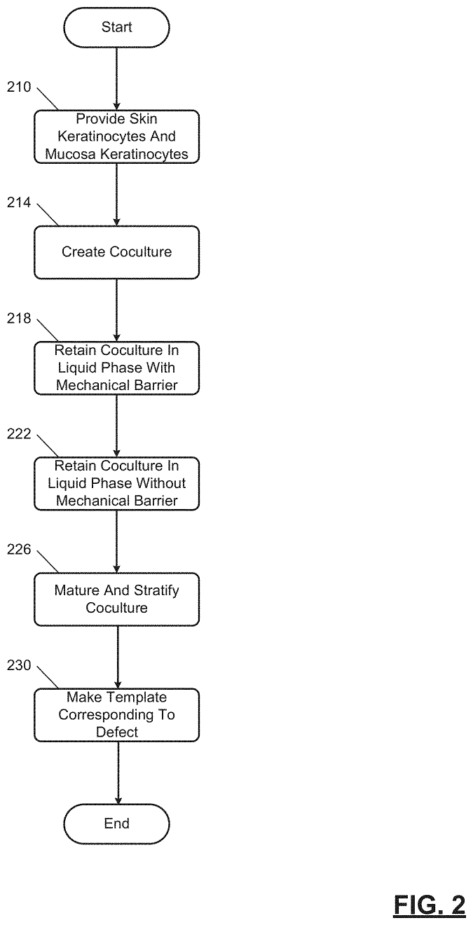

With reference to FIG. 2, a method of creating an MCC in vitro according to various aspects of the present disclosure is provided. The method starts at 210 and generally includes providing skin keratinocytes and mucosa keratinocytes; creating a coculture at 214, retaining the coculture in a first liquid phase with a mechanical barrier at 218; retaining the coculture in a second liquid phase without the mechanical barrier at 222; and maturing and stratifying the coculture at 226 in an air-liquid phase to form the MCC. At 230, the method may further include making a template corresponding to the defect from the MCC. The method ends. Each of these steps is described in greater detail below.

At 210, the method includes providing skin keratinocytes and mucosa keratinocytes. A skin sample is collected via punch biopsy or surgical incision. The skin sample may be obtained from a subject, such as behind the subject's ear. Skin keratinocytes are extracted from the skin sample. Primary skin keratinocyte cultures are created from the skin keratinocytes. The first cell population is created by amplifying a population of the primary skin keratinocyte cultures.

Similarly, a mucosa sample is collected via punch biopsy or surgical incision. In certain aspects, the mucosa sample may be obtained from a subject's oral mucosa, such as in the cheek area. Mucosa keratinocytes are extracted from the mucosa sample. The mucosa keratinocytes may be keratinized or non-keratinized. Primary mucosa keratinocyte cultures are created from the mucosa keratinocytes. The second cell population is created by amplifying a population of the primary mucosa keratinocyte cultures.

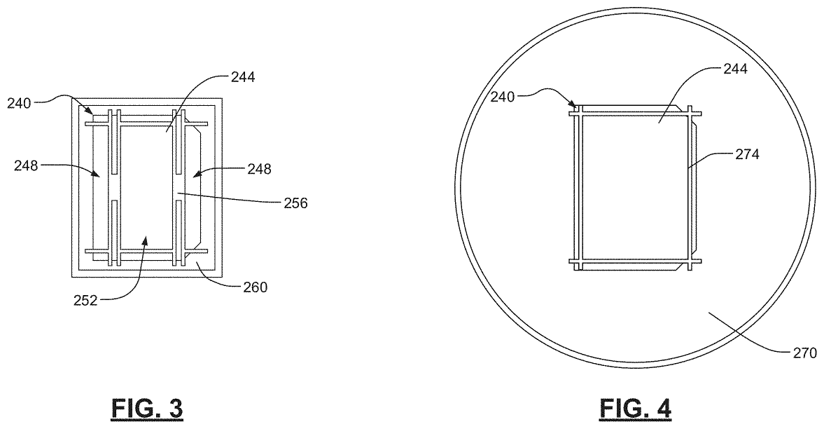

At 214, the method includes creating a coculture. Referring to FIG. 3, a coculture 240 according to various aspects of the present disclosure is provided. The coculture 240 is created on an acellular dermal matrix 244 (also referred to as a “scaffold”), such as decellularized cadaveric human skin. The coculture 240 is created by seeding the first cell population on two first portions 248 of the acellular dermal matrix 244 and seeding the second cell population on one second portion 252 of the acellular dermal matrix 244 between first portions 248. The portions 248, 252 are each separated by a mechanical barrier 256 to reduce or prevent comingling of the first and second cell populations. Moreover, the mechanical barrier 256 creates two cell-free zones where it contacts the acellular dermal matrix 244 between the first and second portions 248, 252. A mechanical barrier may be designed to create different quantities of regions depending on the cell construct to be creates (e.g., two first portions and one second portion to create an MCC for lips, as shown, or one first portion and one second portion to create an MCC for anal sphincter).

At 218 (FIG. 2), the method further includes retaining the coculture in a liquid phase with the mechanical barrier. With continued reference to FIG. 3, the coculture 240 is retained in a first liquid phase 260 for a first predetermined period of time with the mechanical barrier 256 separating the first and second portions 248, 252. In certain aspects, a downward force may be applied to the mechanical barrier 256 to reduce or prevent mingling of cells between the first and second portions 248, 252. The coculture 240 is fully submerged in the first liquid phase 260. In certain aspects, the first period of time is in a range of 18-30 hours (e.g., 24 hours). In certain aspects, the first liquid phase 260 is a serum-free chemically defined medium including calcium.

At 222 (FIG. 2), the method further includes retaining the coculture in the liquid phase without the mechanical barrier. With reference to FIG. 4, the coculture 240 is retained in a second liquid phase 270 for a second predetermined period of time without the mechanical barrier 256 (FIG. 3). After removal of the mechanical barrier 256, the first and second cell populations may migrate toward one another in a transition region corresponding to the cell-free zone. A corral 274 is placed on the acellular dermal matrix 244 to stabilize the acellular dermal matrix 244 in the second liquid phase 270. Accordingly, the coculture 240 is fully submerged in the second liquid phase 270. In certain aspects, the second predetermined period of time may be in a range of 3-5 days (e.g., 4 days). The second liquid phase 270 may be a serum-free chemically defined medium including calcium.

At 226 (FIG. 2), the method further includes maturing and stratifying the coculture to form the MCC. Referring to FIG. 5A, the coculture 240 is raised to an air-liquid interface of an air-liquid phase 280 and retained at the air-liquid interface for a third predetermined period of time. More particularly, the acellular dermal matrix 244 is placed on a permeable membrane insert 282 sitting on top of a riser 284 so that the coculture 240 is disposed at the air-liquid interface (with the permeable membrane insert 282 being between the riser 284 and the acellular dermal matrix 244).

The riser 284 is designed to sink in the air-liquid phase 280. In certain aspects, the riser 284 has a waffle structure including a first plurality of rungs 286-1 extending in a first direction and a second plurality of rungs 286-2 extending in a second direction substantially perpendicular to the first direction (collectively, “the rungs 286”). A portion 288 (FIG. 5B) of the rungs 286, such as two first rungs 286-1, have an increased height compared to the other rungs 286 for minimizing contact with the insert 282.

At the air-liquid interface, a top cellular portion of the coculture 240 is exposed to air while a bottom dermal scaffold portion of the coculture 240 is exposed to liquid. In certain aspects, the third predetermined period is in a range of 8-12 days (e.g., 10 days). During the third predetermined period of time at the air-liquid interface, the skin cells and the mucosa cells develop a normal stratified epithelium. In certain aspects, the medium of the air-liquid phase 280 may be replaced at predetermined intervals during the third predetermined period of time.

The MCC formed after the third predetermined period of time includes a first region 290 including skin cells and a second region 292 including mucosa cells. The first region 290 corresponds to the first portion 248 of the acellular dermal matrix 244 and the second region 292 corresponds to the second portion 252 of the acellular dermal matrix 244. In certain aspects, the MCC further includes a transition region 294 between the first and second regions 290, 292. The transition region 294 corresponds to an area of the acellular dermal matrix where the mechanical barrier 256 (FIG. 3) was initially in contact with the acellular dermal matrix 244.

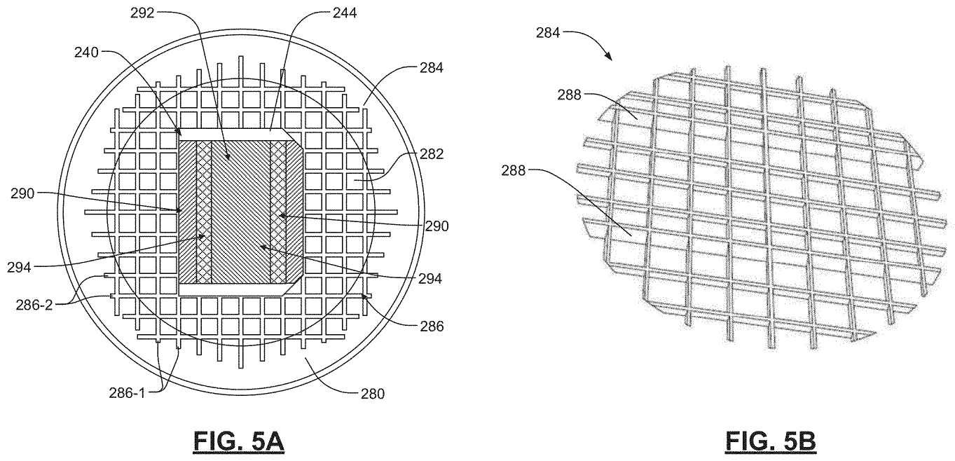

At 230 (FIG. 2), the method may further include making a template corresponding to the defect. With reference to FIG. 6, a template 300 is prepared in vitro from the cell construct, such as the MCC. The template 300 is sized and shaped to correspond to a region of the defect. For example, the template 300 shown in FIG. 6 corresponds to a complete set of lips. The template 300 includes two separate skin regions 304 and one oral mucosa region 308. Each skin region 304 is separated from the oral mucosa region 308 by a respective transitional region 312. A portion of the oral mucosa region 308 is incised or excised to create a stoma 316. Outer portions 320 of the skin and transitional regions 304, 312 are excised at lines 324 to create a shape of the lips. The oral mucosa region 308 is turned or folded in at lines 328 to form a commissure of the lip.

### Forming an Integrated In Vivo Composite

Returning to FIG. 1, at 114, the method includes forming an integrated in vivo composite. Forming the integrated in vivo composite includes isolating the muscle and grafting or implanting the cell construct, such as the MCC, onto a muscle bed. Isolating the muscle includes dissecting the muscle to be free of the underlying connective tissue while preserving the neurovascular bundle pedicle to preserve the viability (perfusion) and innervation of the muscle.

The muscle bed is at a donor site remote from the defect. An appropriate muscle is selected based on the location of the defect. In certain aspects, the muscle is a latissimus dorsi muscle (LDM), a platysma muscle, a gracilis muscle, or any other muscle deemed appropriate for defect site reconstruction. In one example, the LDM is used for reconstruction of lips. In another example the platysma muscle is used for reconstruction of eyelids. In yet other examples, the gracilis muscle is used for reconstruction of a urogenital system, such as a vagina, penis, or anal sphincter.

### Stabilizing the Composite on an Obturator Component

At 118, the method includes stabilizing the composite on an obturator component. In creation of the PIPP free flap, stabilizing is performed at the donor site. Stabilizing generally includes creating a stoma in the muscle, substantially parallel to fibers of the muscle, and inserting at least a portion of an obturator component in the stoma to maintain the stoma. The stoma in the muscle is aligned with the stoma in the cell construct (see, e.g., stoma 316 of FIG. 6).

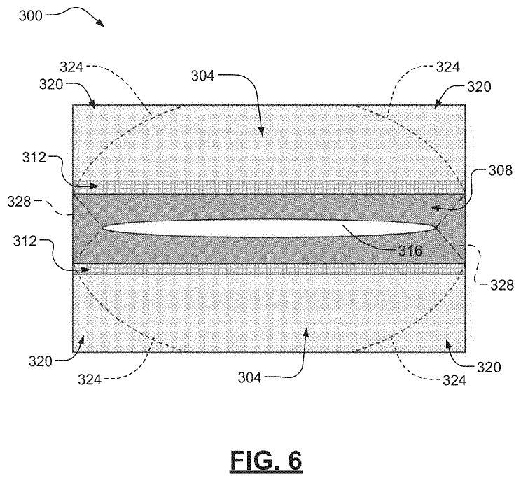

Referring to FIGS. 7-8, an obturator component 350 according to various aspects of the present disclosure is provided. In certain aspects, the obturator component 350 has a single-piece, unitary body that is free of joints and seams. The obturator component 350 may be of solid construction. The obturator component 350 is formed from a material suitable for implantation in the body, such as biocompatible silicone. In certain aspects, the obturator component 350 has substantially smooth surfaces.

The obturator component 350 extends along a longitudinal axis 352. The obturator component 350 generally includes a base portion 354, an obturator portion 358, and an anchor portion 362. The obturator portion 358 extends from the base portion 354 and is disposed between the base portion 354 and the anchor portion 362 along the longitudinal axis 352.

The obturator portion 358 is configured to maintain the stoma in the composite (e.g., in both the muscle and in the cell construct). Therefore, the obturator portion 358 is sized and shaped based on the defect. In certain aspects, the obturator portion 358 is elliptical-cylindrical and has a substantially elliptical cross section perpendicular to the longitudinal axis 352. The elliptical cross section defines a major axis 366 (FIG. 8) perpendicular to the longitudinal axis 352 and a minor axis 370 (FIG. 8) perpendicular to the major axis 366 and the longitudinal axis 352. In certain other aspects, the obturator portion 358 may define other cross-sectional shapes, such as substantially circular.

The obturator portion 358 further defines a height 374 (FIG. 7) parallel to the longitudinal axis 352 and between the base portion 354 and the anchor portion 362. The height 374 is selected to accommodate a combined thickness of the muscle and the cell construct.

The base portion 354 is configured to engage an interior face of the muscle to reduce or prevent displacement of the obturator component 350 with respect to the composite. In certain aspects, the base portion 354 may be referred to as a planar base portion because its thickness (i.e., parallel to the longitudinal axis 352) is much less than each of its length and width. The base portion 354 may have a substantially constant thickness a substantially planar top surface 378 and a substantially planar bottom surface 382. In certain aspects, the base portion 354 is substantially cylindrical.

The anchor portion 362 is configured to cooperate with the base portion 354 to maintain the cell construct in direct opposition to the muscle. The anchor portion 362 is also configured to reduce or prevent displacement of the obturator component 350 with respect to the composite. In certain aspects, the anchor portion 362 defines a substantially rectangular prism shape. However, in certain other aspects, the anchor portion 362 may define other shapes, such as other elongated shapes.

The anchor portion 362 may be an elongated anchor portion 362 extending along a major axis 386 (FIG. 8) that is substantially perpendicular to the longitudinal axis 352. The major axis 386 of the anchor portion 362 extends nonparallel to the major axis 366 of the obturator portion 358. In certain aspects, the major axis 386 of the anchor portion 362 extends substantially perpendicular to the major axis 366 of the obturator portion 358.

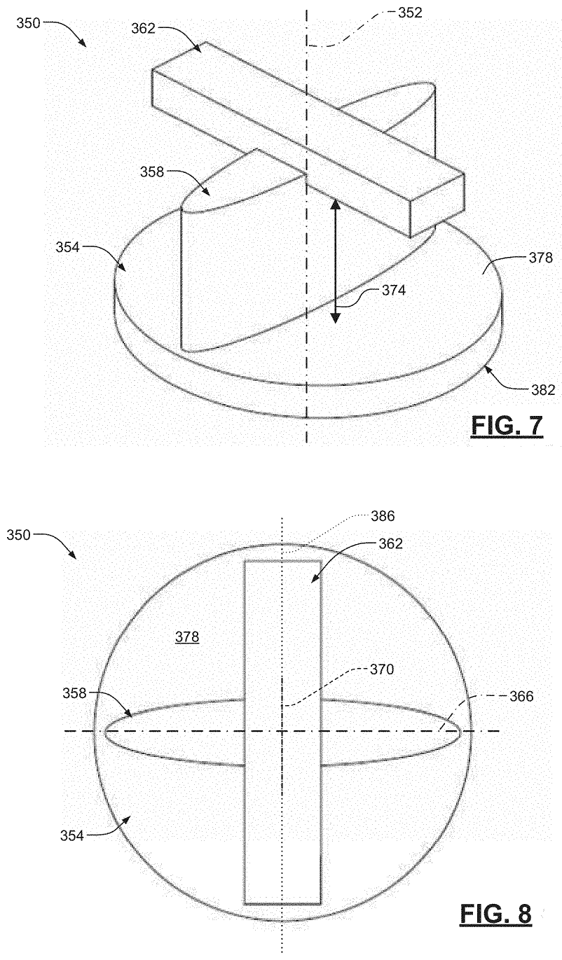

With reference to FIG. 9, a composite 410 is stabilized on the obturator component 350 according to various aspects of the present disclosure. The obturator component 350 may be stabilized to the composite 410 by its physical engagement with the composite 410. More particularly, the composite 410 is disposed between the base portion 354 and the anchor portion 362, with the obturator portion 358 (FIGS. 7-8) extending through and maintaining a stoma in the composite 410. A portion of the obturator portion 358 adjacent to the anchor portion 362 may project from the stoma. The composite 410 is oriented such that a muscle 414 is disposed adjacent or closest to the base portion 354 and a cell construct 418 is disposed adjacent or closest to the anchor portion 362. In certain aspects, a biocompatible sheet 422 (e.g., SILASTIC sheet) is disposed between each of the cell construct 418 and the anchor portion 362 and the muscle 414 and the base portion 354.

The base portion 354 and the anchor portion 362 cooperate to stabilize the composite 410 in between the base portion 254 and the anchor portion 362. Accordingly, the cell construct 418 is maintained in substantially continuous contact with the muscle 414. The continuous contact may reduce or prevent the occurrence of blood clots that would inhibit revascularization between the muscle 414 and the cell construct 418. In certain aspects, the base portion 354 is shaped and sized to maximize engagement with the muscle 414 and stabilize the obturator component 350 against the muscle 414. In certain aspects, the anchor portion 362 is sized and shaped to minimize engagement with the cell construct 418.

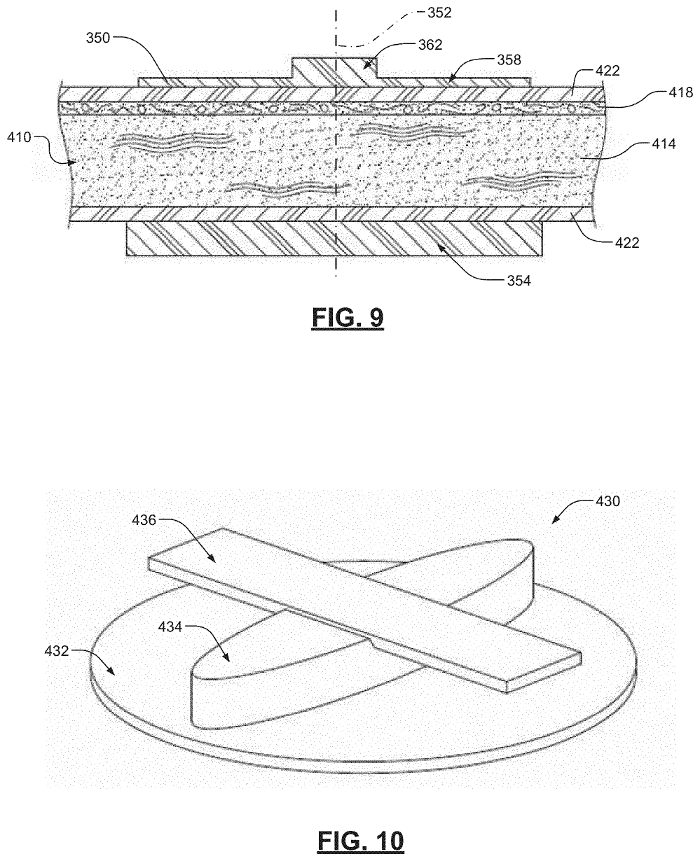

Obturators may be made in custom sizes and/or geometric shapes to accommodate tissues for a particular defect, or in bulk for common procedures. In certain aspects, the obturator component 350 of FIGS. 7-9 is sized and shaped to reconstruct human lips. Referring to FIG. 10, another obturator component 430 according to various aspects of the present disclosure is provided. The obturator component 430 includes a base portion 432, an obturator portion 434, and an anchor portion 436, which have similar function to the base portion 354, obturator portion 358, and anchor portion 362 of the obturator component 350 of FIGS. 7-9, respectively, but are sized and shaped for reconstruction of a human eye.

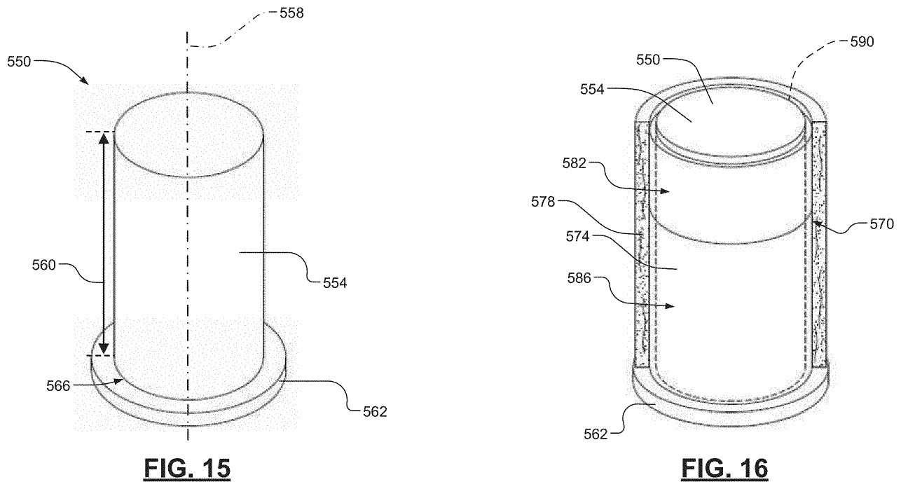

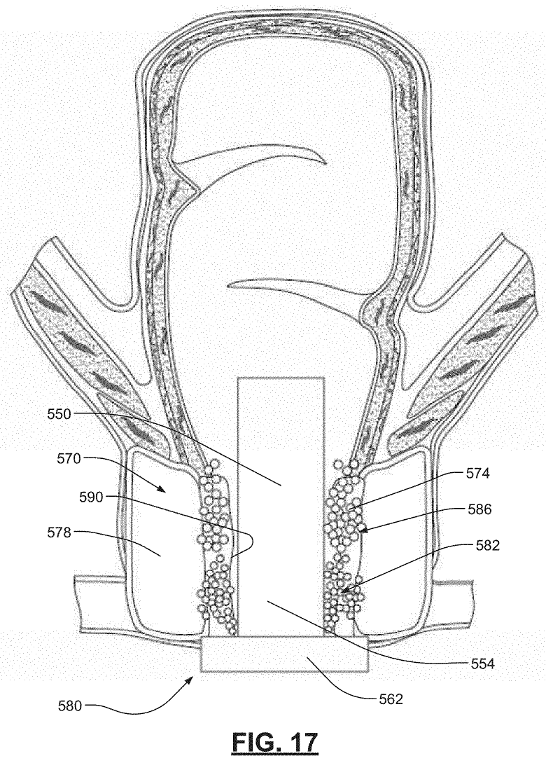

In other aspects an obturator component according to various aspects of the present disclosure includes different or additional portions than those described above. For example, an obturator component may include an obturator portion having a circular, elliptical, or other shaped cross section for maintaining a stoma, while omitting base and/or anchor portions (see, e.g., the obturator component 550 of FIGS. 15-16).

### Developing a Microvascular System

Returning to FIG. 1, at 122, the method includes developing a microvascular system in the composite. Developing a microvascular system includes retaining the composite stabilized on the obturator component in vivo for a predetermined period of time. In certain aspects, the predetermined period of time is in a range of 10-20 days (e.g., 14 days). During the predetermined period of time, the cell construct, such as the MCC, matures, integrates with the muscle, and develops a microcapillary system with the underlying muscle. More specifically, the muscle functions as an in situ bioreactor for the implanted cell construct by allowing vessel ingrowth to develop the microcapillary system.

### Removing the Obturator Component

With continued reference to FIG. 1, at 124, the obturator component is removed from the stoma of the composite. The stoma remains in the composite after the obturator component is removed.

### Transferring Composite to Recipient Site of Defect

At 126, the method further includes transferring the composite to a recipient site of the defect based on a native axial blood supply at the recipient site. Transferring generally includes harvesting a PIPP free flap by surgically removing the composite from the donor site and implanting the free flap to the recipient site. Implanting includes micro-anastomosing neurovascular pedicles in the PIPP free flap to vascular structures (artery and vein) the recipient site and the motor nerve at the recipient site. Transferring may further include modifying a shape of the PIPP flap prior to attaching the PIPP flap at the recipient site of the defect to match the defect to be repaired. The shape is modified to correspond to the defect, for example, an upper lip, a lower lip, or a portion of the upper lip and/or lower lip.

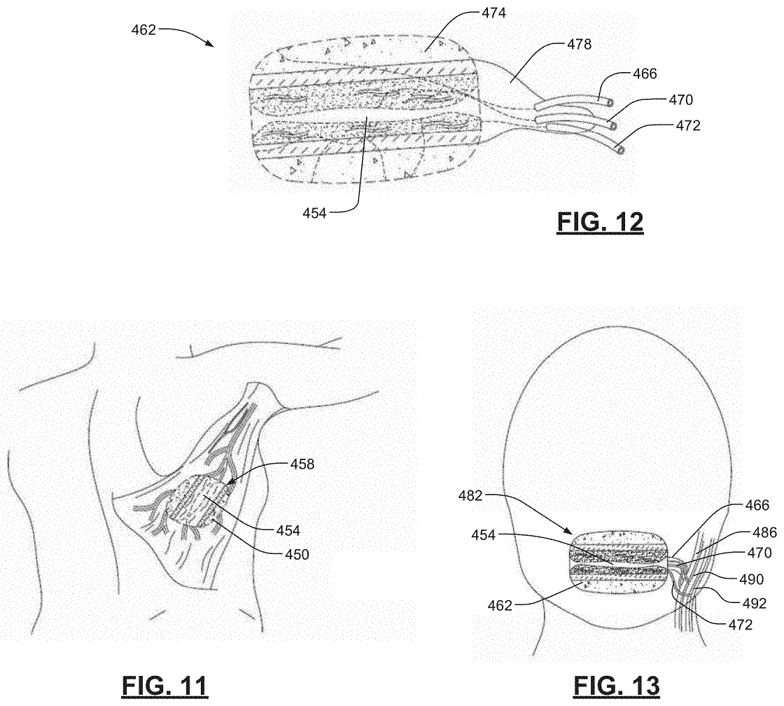

Referring to FIG. 11, a composite 450 including a surgically-created stoma 454 at a donor site 458 according to various aspects of the present disclosure is provided. In certain aspects, the donor site 458 includes the LDM. With reference to FIG. 12, a PIPP free flap 462 including the stoma 454 is created by surgically removing the composite 450 from the donor site 458. The PIPP free flap 462 includes an artery pedicle 466, a vein pedicle 470, and a nerve pedicle 472 (e.g., from the thoracodorsal artery, vein, and nerve when the donor site includes the LDM). The PIPP free flap 462 further includes the cell construct 474 (e.g., the MCC) and the muscle 478.

Referring to FIG. 13, the PIPP free flap 462 is implanted at a recipient site 482 of the defect, such as to form all or a portion of lips. The artery and vein pedicles 466, 470 of the PIPP free flap 462 are micro-anastomosed to respective artery and vein pedicles 486, 490 at the recipient site 482 (e.g., the external carotid (or facial) artery and external jugular (or facial) vein when the defect includes lips). The nerve pedicle of the PIPP free flap 462 is micro-anastomosed to a motor nerve 492 at the recipient site 482 (e.g., the thoracodorsal nerve is anastomosed to the facial nerve or one of its branches, in the case of LDM donor site and lip defect) for motor nerve function.

In certain aspects, after the PIPP free flap 462 is implanted at the recipient site 482, sensory innervation may be partially or fully restored in the defect. For example, sensory innervation may be gradually restored over a period of time via an ingrowth of sensory nerves.

### Repairing a Soft Tissue Defect with a PIPP Rotational Flap

With reference to FIG. 14, a method of repairing a soft tissue defect with a PIPP rotational flap according to various aspects of the present disclosure is provided. The method begins at 510 by providing a cell construct. At 514, the method includes forming an integrated in vivo composite. At 518, the method further includes developing a microvascular system in the composite in situ. At 522, the method further includes transferring the composite to a recipient site of a defect. At 526, the method further includes stabilizing the composite on an obturator component. At 530, the method further includes removing the obturator component from the composite.

### Providing Cell Construct

Providing the cell construct at 510 is the same as or similar to providing the cell construct at 110 of FIG. 1, described above.

### Forming Integrated In Vivo Composite

Forming the integrated in vivo composite at 514 is the same as or similar to forming the integrated in vivo composite at 114 of FIG. 1. When repairing a defect using a rotational flap, an appropriate muscle is proximate to the defect site so that the muscle remains attached to its neurovascular pedicle when transferred from a donor site to the recipient site of the defect. For example, the gracilis muscle may be a suitable donor site for reconstruction of portions of the urogenital system, such as a vagina or anal sphincter.

### Developing Microvascular System in Composite

At 518, the method includes developing a microvascular system in the composite. Development of the microvascular system is the same as or similar to development of the microvascular system at 126 of FIG. 1, described above.

### Transferring Composite to Recipient Site of Defect

At 522, the method further includes transferring the composite to a recipient site of the defect. Transferring the composite to the recipient site includes rolling the composite from the donor site to the recipient site while the neurovascular pedicle of the muscle remains intact to form a lumen in which to place the obturator.

### Stabilizing Composite on an Obturator Component

At 526, the method includes stabilizing the composite on an obturator component. The stabilizing is performed at the recipient site of the defect. Stabilizing generally includes surgically creating a rotational flap including the composite and rolling at least a portion of the rotational flap around at least a portion of the obturator component to at least partially define a stoma or lumen with the obturator extending through the stoma or lumen.

Referring to FIG. 15, an obturator component 550 according to various aspects of the present disclosure is provided. In certain aspects, the obturator component 550 has a single-piece, unitary body that is free of joints and seams. The obturator 550 may have a solid construction. The obturator component 550 is constructed from a material suitable for implantation in the body, such as biocompatible silicone. The obturator component 550 may have surfaces that are substantially smooth.

The obturator component 550 includes a body 554. The body 554 may be an elongated body extending along a longitudinal axis 558. The body 554 defines a length 560 substantially parallel to the longitudinal axis 558. In certain aspects, such as when the obturator component 550 is to be used to define a lumen in a vagina or anal sphincter, the body 554 defines a substantially cylindrical shape. The obturator component 550 may further include a flange 562 disposed at an end 566 of the body 554 and extending radially outwardly from the body 554. The flange 562 is configured to facilitate stabilization of the obturator component 550 in the stoma or lumen.

With reference to FIGS. 16-17, a composite 570 stabilized on the obturator component 550 according to various aspects of the present disclosure is provided. FIG. 16 depicts the composite 570 in partial cutaway and FIG. 17 depicts the composite 570 at a recipient site 580 of a defect. In certain aspects, the recipient site 580 includes an anal sphincter.

The composite 570 includes a cell construct 574 and a muscle 578, with the cell construct 574 being disposed between the obturator component 550 and the muscle 578. In certain aspects, the cell construct 574 is an MCC including a first region 582 including skin cells and a second region 586 including mucosa cells. The cell construct 574 may be directly disposed on the body 554 of the obturator component 550 or have a silicone sheet (not shown) disposed between the obturator component 550 and the cell construct 574. The composite 570 may be wrapped around a portion of a circumference and/or length the body 554 or substantially the entire circumference and/or length of the body 554 (e.g., wrapped around the entire circumference to form a cylindrical lumen 590). The flange 562 may project from the created cavity or lumen 590 to facilitate retention of the obturator component 550 in the cavity or lumen 590.

The obturator component 550 is retained in the stoma or lumen for a predetermined period of time. The predetermined period of time may be in a range of 10-20 days (e.g., 14 days).

### Removing Obturator Component From Composite

At 530, the method further includes removing obturator component from the stoma or lumen of the composite. The composite retains the stoma or lumen after removal of the obturator component. The PIPP rotational flap formed from the composite remains at the recipient site of the defect to restore damaged tissue of the defect.

In certain aspects, sensory innervation may be partially or fully restored in the defect. For example, sensory innervation may be gradually restored over a period of time via an ingrowth of sensory nerves.

### Example 1

Methods according to various aspects of the present disclosure are performed on athymic rats.

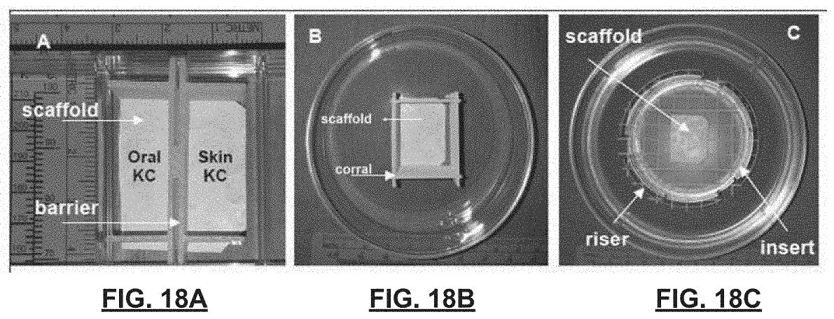

First, an MCC is prepared. A 3×3 cm acellular dermal scaffold (ADM) is washed in 1× Dulbecco's phosphate-buffered saline (DPBS) three times for 15 minutes/time. The DPBS is changed for each wash. The scaffold is submerged in 1×DPBS overnight. Scaffolds are coated with 0.05 mg/ml human type IV collagen at 4° C., overnight. Collagen solution is aspirated completely, followed by rinsing the scaffold with medium. After aspiration of the medium a barrier is placed on top of scaffold and pressed firmly onto the scaffold. Oral and skin keratinocytes are each seeded at 500K cells/cm² in medium (concentration optimized from previous studies) containing 0.06 mM calcium onto respective chambers or portions, as shown in FIG. 18A, having one cell-free zone in between (where the barrier contacts the scaffold). Cells are incubated inside a 5% CO incubator at 37° C. for 24 h. Medium is aspirated completely before the removal of the barrier. The scaffold is then transferred to a 100×20 mm dish, a corral is placed on top of the scaffold to pin it down and prevent its movement/floating, as shown in FIG. 18B. Then, 30 ml medium containing 1.2 mM calcium is added to continue a liquid phase culture for 4 days to stimulate differentiation. The medium is changed every day. After 4 days, the scaffold is transferred onto a 75 mm diameter insert sitting on top of a riser inside a 150×25 mm cell culture dish, as shown in FIG. 18C, with enough medium containing 1.2 mM calcium to create an air-liquid phase to initiate cell stratification. The medium is changed every two days for 10 days. The MCC is then cut into 3 strips with the dimensions of 30×8 mm. One strip is evaluated by immunohistochemistry and the remaining two are used for implantation into athymic rats. A control group without cells of ADM is processed in a similar manner to the scaffold with cells to manufacture the MCC.

Second, the MCCs are surgically implanted into rats. A 6 cm dorsal incision overlying the latissimus dorsi muscle (LDM) of rats is made from head to tail. The LDM is then isolated and dissected free of the underlying connective tissue with preservation of the neurovascular bundle nerve to preserve the viability and innervation of the muscle. At this time the ADM without cells or MCCs with cells are grafted onto the muscle bed. A circular piece of gas sterilized biomedical-grade silicone sheeting, 0.005 in. thick, placed above and below the grafts to prevent adherence of the epithelial layers of the tissue composite to the connective tissues of the subcutaneous pouch. The open reticular or dermal portion of the ADM or MCC is grafted, dermal side down, with the epithelia side (cell keratinocyte side) facing up, onto the muscular fascia to allow ingrowth of a microvascular capillary network. Then, an opening is made in the center of the ADM or MCC by separating the muscle fibers, longitudinally, through the entire muscle to simulate a stoma or opening. An obturator is placed in the surgically created opening to maintain the stoma.

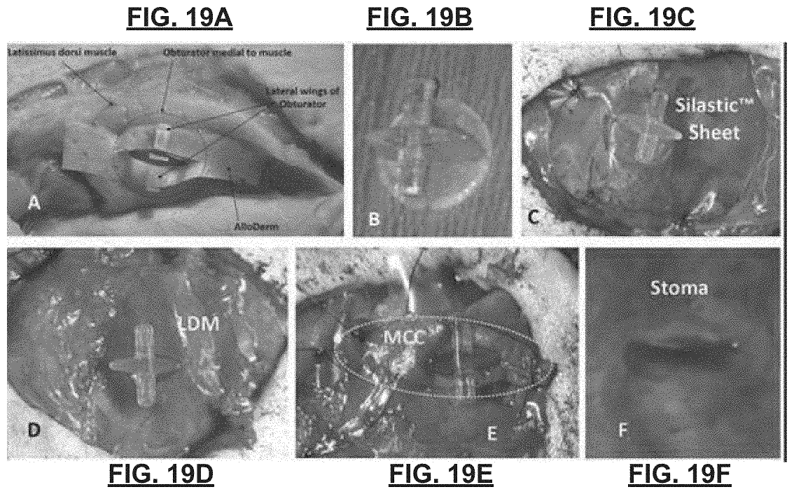

FIGS. 19A-19F depict portions of a method according to various aspects of the present disclosure. FIG. 19A depicts a laminate (e.g., AlloDerm) an obturator in situ in a cadaveric rat. FIG. 19B depicts the obturator. FIG. 19C depicts the obturator in place with a SILASTIC sheet and the obturator sutured in place to underlying subcutaneous tissue (SQ) tissue. FIG. 19D depicts the LDM placed over the obturator through a surgically-created stoma in the muscle. FIG. 19E depicts the MCC placed over the obturator on the LDM and sutured in place. FIG. 19F depicts the stoma maintained, healed, and functional after removal of the obturator.

### Example 2

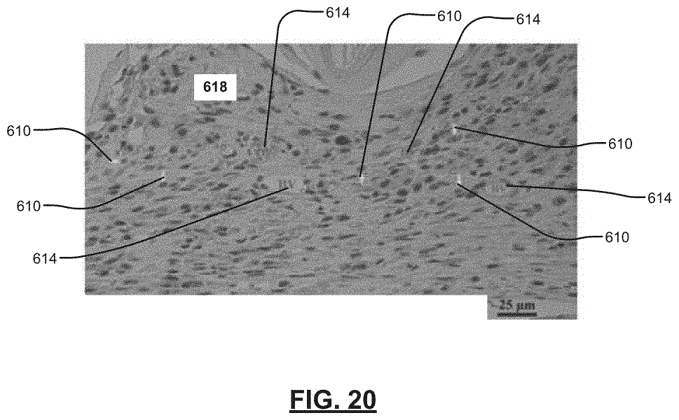

An ex vivo-produced oral mucosa equivalent (EVPOME) is provided. The EVPOME is implanted into an SCID mouse. Four weeks post implantation, the appearance of potential neurons is detectable by using anti-NeuN antibody, as shown in FIG. 20. The potential neurons are shown at 610. Blood vessels are shown at 614. A cross section of the EVPOME is shown at 618. The potential neurons are believed to indicate restoration of sensory innervation that can occur over an extended period of time. Sensory Innervation is responsible for touching, temperature, or sensational feelings.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of forming a prefabricated innervated pre-vascularized pre-laminated (PIPP) flap having a stoma or lumen, the method comprising:
providing a cell construct including skin cells, mucosa cells, or both skin cells and mucosa cells;
forming an integrated in vivo composite at a donor site by grafting the cell construct onto a muscle;
stabilizing the composite on an obturator component, the obturator component being configured to maintain a stoma or lumen in the composite or formed by the composite;
developing a microvascular system in the composite by retaining the composite in vivo at the donor site for a first predetermined period of time; and
removing the obturator component from the stoma or lumen.

2. The method of claim 1, wherein
the stabilizing is performed at the donor site,
the developing includes retaining the composite and the obturator component in vivo at the donor site for the first predetermined period of time, and
the method further comprises harvesting the PIPP flap by surgically removing the composite from the donor site, the PIPP flap being a free flap.

3. The method of claim 2, wherein
the stoma or lumen is the stoma, and
the stabilizing includes
creating the stoma in the muscle, the stoma being substantially parallel to strata of fibers of the muscle, and
inserting at least a portion of the obturator component into the stoma to maintain the stoma.

4. The method of claim 3, wherein the obturator component includes
a planar base portion defining a longitudinal axis, the longitudinal axis being substantially perpendicular to a plane of the planar base portion,
an obturator portion extending from the planar base portion, the obturator portion defining a first axis perpendicular to the longitudinal axis, and
an elongated anchor portion extending from the obturator portion, the elongated anchor portion defining a second axis perpendicular to the longitudinal axis and nonparallel to the first axis, the obturator portion being between the planar base portion and the elongated anchor portion along the longitudinal axis, wherein the obturator portion is configured to maintain the stoma.

5. The method of claim 1, further comprising:
surgically creating a PIPP rotational flap including the composite such that a neurovascular pedicle of the muscle of the composite remains intact, wherein
the stoma or lumen is the lumen, and
the stabilizing is performed at a recipient site of a defect and includes rolling the rotational flap around at least a portion of a length of the obturator component to define at least a portion of the lumen.

6. The method of claim 5, wherein the obturator component includes an elongated body defining the length.

7. The method of claim 6, wherein the obturator component further includes a flange.

8. The method of claim 1, wherein the cell construct is a mucocutaneous construct (MCC) including a first region including skin cells and a second region including mucosa cells.

9. The method of claim 8, wherein the providing includes creating the MCC in vitro.

10. The method of claim 9, wherein the creating includes,
providing a first population of skin keratinocytes and a second population of mucosa keratinocytes,
creating a coculture by seeding the first population and the second population on a respective first portion and second portion of a non-immunogenic acellular dermal matrix in a first liquid phase, the first portion and the second portion being separated by a mechanical barrier,
retaining the coculture with the mechanical barrier in the first liquid phase for a second predetermined period of time,
removing the mechanical barrier and retaining the coculture in a second liquid phase for a third predetermined period of time, and
maturing and stratifying the coculture by retaining the coculture in an air-liquid phase for a fourth predetermined period of time, thereby forming the MCC.

11. The method of claim 10, wherein the providing the first population includes
collecting a skin sample via punch biopsy or surgical incision,
extracting skin keratinocytes from the skin sample,
creating primary skin keratinocyte cultures from the skin keratinocytes, and
amplifying a population of the primary skin keratinocyte cultures.

12. The method of claim 10, wherein the providing the second population includes
collecting a mucosa sample via punch biopsy or surgical incision,
extracting mucosa keratinocytes from the mucosa sample,
creating primary mucosa keratinocyte cultures from the mucosa keratinocytes, and
amplifying a population of the primary mucosa keratinocyte cultures.

13. The method of claim 11, wherein the mucosa keratinocytes are keratinized or non-keratinized oral mucosa keratinocytes.

14. The method of claim 1, wherein the muscle is a latissimus dorsi muscle (LDM), a platysma muscle, or a gracilis muscle.

15. The method of claim 1, wherein the first predetermined period of time is in a range of 10 days to 20 days.

16. A method of restoring a defect including damaged or surgically removed soft tissue, the method comprising:
providing a cell construct including skin cells, mucosa cells, or both skin cells and mucosa cells;
forming an integrated in vivo composite at a donor site by grafting the cell construct onto a muscle;
stabilizing the composite on an obturator component, the obturator component being configured to maintain a stoma or lumen in the composite or formed by the composite;
developing a microvascular system in the composite by retaining the composite in vivo at the donor site for a predetermined period of time;
removing the obturator component from the stoma or lumen; and
transferring the composite to a recipient site of the defect.

17. The method of claim 16, wherein the recipient site and the donor site are on the same human or animal.

18. The method of claim 17, wherein the skin cells, the mucosa cells, or both the skin cells and the mucosa cells are grown from the same human or animal.

19. The method of claim 16, further comprising:
harvesting a PIPP free flap by surgically removing the composite from the donor site, wherein
the stabilizing is performed at the donor site prior to the developing,
the developing includes retaining the composite and the obturator component in vivo at the donor site for the predetermined period of time, and
the transferring includes micro-anastomosing neurovascular pedicles in the PIPP free flap to respective vascular pedicles and a motor nerve at the recipient site.

20. The method of claim 16, further comprising:
after the developing, surgically creating a PIPP rotational flap including the composite such that a neurovascular pedicle of the muscle of the composite remains intact, wherein
the stoma or lumen is the lumen,
the transferring includes rotation of the PIPP rotational flap around the neurovascular pedicle from the donor site to the recipient site, and
the stabilizing is performed at the recipient site and includes rolling the PIPP rotational flap around at least a portion of a length of the obturator component to form at least a portion of the lumen.

21. The method of claim 16, wherein the transferring includes modifying a shape, a size, or both a shape and size of the composite based on the recipient site.

22. The method of claim 16, wherein the cell construct is a mucocutaneous construct (MCC) including a first region including skin cells and a second region including mucosa cells.

23. The method of claim 16, wherein the damaged soft tissue includes at least a portion of lips, an eyelid, an ear, a nose, a vagina, penis, or an anal sphincter.

24. The method of claim 16, wherein the providing includes creating the MCC in vitro.

25. An obturator component for forming a prefabricated innervated pre-vascularized pre-laminated (PIPP) free flap having a stoma, the obturator component comprising:
a planar base portion defining a longitudinal axis, the longitudinal axis being substantially perpendicular to a plane of the planar base portion;
an obturator portion extending from the planar base portion, the obturator portion defining a first axis perpendicular to the longitudinal axis; and
an elongated anchor portion extending from the obturator portion, the elongated anchor portion defining a second axis perpendicular to the longitudinal axis and nonparallel to the first axis, the obturator portion being between the planar base portion and the elongated anchor portion along the longitudinal axis, wherein the obturator portion is configured to maintain the stoma.

26. The obturator component of claim 25, wherein the first axis is substantially perpendicular to the second axis.

27. The obturator component of claim 25, wherein the planar base portion is substantially cylindrical.

28. The obturator component of claim 25, wherein the obturator portion is substantially elliptical cylindrical and the first axis is a major axis of the ellipse.

29. The obturator component of claim 25, wherein the elongated anchor portion is substantially a rectangular prism.

30. The obturator component of claim 25, wherein the obturator component comprises a unitary structure comprising biocompatible silicone.
